(19) United States
(12) Patent Application Publication
(10) Pub. No.: US 2016/0271311 A1
(43) Pub. Date: Sep. 22, 2016

(54) DEVICE WITH A FLUID PUMP, AT LEAST TWO POINTS OF ACCESS TO THE ABDOMINAL WALL AND TUBES CONNECTING THE FLUID PUMP AND ABDOMINAL WALL

(71) Applicant: novalung GmbH, Heilbronn (DE)

(72) Inventor: Georg MATHEIS, Heilbronn (DE)

(73) Assignee: novalung GmbH, Heilbronn (DE)

(21) Appl. No.: 15/034,202

(22) PCT Filed: Oct. 24, 2014
(86) PCT No.: PCT/DE2014/000540

§ 371 Date: May 4, 2016

(30) Foreign Application Priority Data
Nov. 5, 2013 (DE) 10 2013 018 444.4

(51) Int. Cl. A61M 1/28; A61M 1/32

(52) U.S. Cl. CPC A61M 1/285; A61M 1/32; A61M 1/287

(57) ABSTRACT

A device with a blood pump, at least two points of access to the abdominal wall and tubes connecting the blood pump and points of access to the abdominal wall, which is known as a peritoneal dialysis system, is equipped with a CO₂ removal arrangement, in order to use it tor blood oxygenation and/or CO₂ removal.

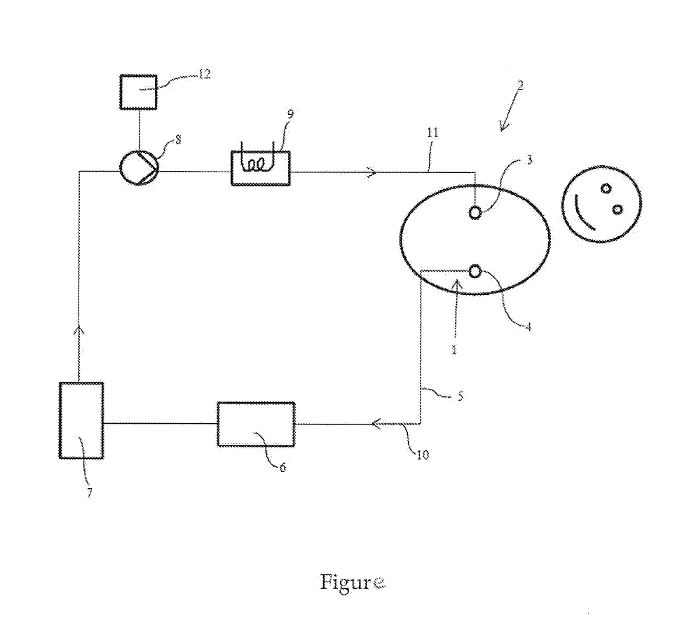

The invention relates to a device with a fluid pump, at least two points of access to the abdominal wall and tubes connecting the fluid pump and abdominal wall.

Such devices are known for example as peritoneal dialysis systems. Peritoneal dialysis serves for intracorporeal blood purification. In peritoneal dialysis, a volume of approximately 1 to 3 litres diaiysate, which consists of an electrolyte solution, is applied into the peritoneal cavity for the detoxification of the blood. The transport of toxic metabolites, but also electrolytes, buffer substances and water from the blood of the patient via the peritoneal membrane into the dialysate takes place by diffusion. The removal of the harmful substances from the organism therefore takes place by a drainage of the dialysate to the exterior. Peritoneal dialysis is used in renal insufficiency and can be carried out independently of a dialysis centre.

In order to produce a permanent access to the peritoneal cavity, generally a peritoneal dialysis catheter made of silicone is used.

The invention is based on the problem of constructing such a device in such a way that it can also be used for other fields of medical use.

This problem is solved by a generic device which has a CO₂ removal arrangement, which has a fluid connection with the fluid pump and with the access points to the abdominal wall, in order to remove CO₂ from a fluid.

The simple use of a CO₂ removal arrangement in connection with an arrangement which is known from the field of peritoneal dialysis enables the support of the gas exchange in the case of a damaged lung or respiratory problems.

The device known from dialysis can therefore also be used for CO₂ removal. Whilst the known device supports the kidney, the new device supports the lung. According to the invention, a device which is known from the field of blood purification is therefore modified such that it is able to be used for a gas exchange in relation to the blood.

It is advantageous if the device has a temperature control arrangement, which has a fluid connection with the fluid pump and the CO₂ removal arrangement. Such a temperature control arrangement, which heats or cools a stream of liquid or of gas flowing into the abdominal cavity, can be used as sole arrangement in the fluid stream or can be constructed as a combination arrangement in connection with the fluid pump or with the CO₂ removal arrangement.

Cumulatively or alternatively it is proposed that the tubes are temperature-controlled. Tubes which are able to be temperature-controlled can therefore cool or heat the fluid and thereby offer the attending doctor a large spectrum of effect on a human or animal body.

So that the attending doctor is always informed in an ongoing manner of the composition of the fluid which is used, a measuring arrangement is proposed for determining the fluid composition. This measuring arrangement can also be connected to a control arrangement, in order to act on the fluid composition in a regulating manner. In the simplest case, the temperature of the fluid is regulated by the measuring arrangement. However, the concentration of gases such as CO₂ or O₂ in the fluid or else the content of electrolytes, buffer substances etc. can be measured and, if applicable, also regulated.

The fluid can be a gas or a vapour which is directed through the abdominal cavity. Different gas or vapour compositions are suitable, according to the case of application, wherein preferably humidified gases are used.

However, it is particularly advantageous if the fluid is a liquid. Such a liquid is suitable particularly for CO₂ removal, because it provides for a gas transition from the blood into the liquid in the region of the abdominal cavity, in order for example to remove CO₂ from the blood.

In particular when the fluid is a liquid, it is advantageous if the device has a control for the pump. This makes it possible either to provide a very constant fluid flow or else a pulsating stream, which correlates with the movements of the diaphragm or improves the gas transition by a pressure modulation.

In order to improve the effectiveness of the device, it is proposed that it has a fluid with CO₂-binding substances, such as perfluorocarbons for example. Different CO₂-binding substances which are able to be used medically are known, which depending on the purpose of use can be introduced into the fluid in order to intensify the CO₂ removal.

A simple variant embodiment makes provision that the CO₂ removal arrangement is an oxygenator. A membrane lung or a membrane ventilator can serve as CO₂ removal arrangement. In practical use, it has been found that bubble oxygenators and membrane oxygenators are also very suitable. In particular, membrane oxygenators can be used here differently from usual, if two liquids or gas and liquid flow through them in order to transfer CO₂ from one liquid into another liquid.

For several medical applications, it is advantageous if the CO₂ removal arrangement has an oxygen enrichment arrangement. Such an oxygen enrichment arrangement can operate either via ambient air, via oxygen-enriched gases or with almost pure oxygen.

It is advantageous if the points of access to the abdominal wall are implantable, closable channels. This makes it possible that the patient can use these points of access to the abdominal wall repeatedly for the treatment of his blood.

It has proved to be particularly advantageous here if the points of access to the abdominal wall are permanently elastic, biocompatible sockets for superficial stitching or gluing.

It is particularly simple if known peritoneal dialysis catheters are used as points of access to the abdominal wall.

It is advantageous here if the points of access to the abdominal wall have tubes with lateral openings.

A particularly advantageous variant embodiment of a point of access to the abdominal wall makes provision that at least two points of access to the abdominal wall are constructed such that they are able to be arranged in one abdominal wall channel. This makes it possible, for example, to direct the inflow and the outflow via one via one point of access to the abdominal wall, wherein preferably the inflow and outflow are arranged radially in one another. For this, for example, an inner tube with a smaller diameter can be arranged in an outer tube with a larger diameter, so that a fluid can be pumped into the abdominal cavity via the inner tube with a smaller diameter, that thereafter can be discharged from the abdominal cavity again via the intermediate space between the outer and inner tube. Pumps can be controlled here as pressure and suction pumps so that the pressure of the fluid onto the boundary of the abdominal cavity does not exceed a maximum value. Furthermore, it is also advantageous to take care, via a regulation, that the pressure in the abdominal cavity does not fail below a minimum value.

The problem forming the basis of the invention is also solved by a method for operating such a device, in which a fluid is directed through the fluid pump, the points of access to the abdominal wall and a CO₂ removal arrangement, in order to remove CO₂ from a fluid. Here, the abdominal cavity can firstly be filled with fluid and subsequently emptied. Preferably, the fluid is delivered and discharged continuously through the points of access to the abdominal wall According to the case of application, the flow parameters can be varied, in order for example to achieve a pulsatile perfusion.

The problem forming the basis of the invention is also solved by a method for the use of points of access to the abdominal wall, in which a fluid is pumped through the points of access to the abdominal wall and the fluid is directed through a CO₂ removal arrangement.

An example embodiment of a device according to the invention within the framework of its use on the patient is shown in the FIGURE and is explained in further detail below.

There is shown

The FIGURE: the device with CO₂ removal arrangement in use on the patient

Two permanently elastic, biocompatible sockets 3, 4 are stitched superficially, as point of access to the abdominal wall, in the abdomen 1 of a patient 2. These points of access to the abdominal wall are constructed as peritoneal dialysis catheters and make it possible to fill a fluid 5 into the abdomen 1 of the patient via the point of access 3 and to remove it from the abdomen 1 again via the point of access 4. This liquid is directed here via a measuring arrangement 6 for determining the fluid composition and is subsequently fed to a CO₂ removal arrangement 7. From there, the liquid 5 arrives at a fluid pump 8 and then via a temperature-control arrangement 9 to the access to the abdominal wall 3, through which the liquid arrives back into the abdomen again.

The liquid is directed here through lines 10, 11 (only numbered by way of example), which can also be embodied so as to be heated. According to the intended use, the temperature-control arrangement 9 and the tube heating can also be used for a fluid cooling.

To control the pump 8, a pump control 12 is provided, which can not only switch the pump on and off but can vary the pumped volume and can also vary the pumped quantity chronologically.

The described simple construction enables an application for oxygen enrichment of the blood of a patient with simultaneous CO₂ removal and temperature control of the blood via the abdomen.

1. A device with a fluid pump (8), at least two points of access (3, 4) to the abdominal wall and tubes (10, 11) connecting fluid pump (8) and points of access has a CO₂ removal arrangement (7), which has a fluid connection with the fluid pump (8) and the points of access (3, 4) to the abdominal wall, in order to remove CO₂ from a fluid (5).

2. The device according to claim 1, characterized in that wherein it has a temperature-control arrangement (9), which has a fluid connection with the fluid pump (8) and the CO₂ removal arrangement (7).

3. The device according to claim 1, wherein the tubes (10, 11) are temperature-controlled.

4. The device according to claim 1, wherein it has a measuring arrangement (6) for determining the fluid composition.

5. The device, according to claim 1, wherein the fluid (5) is a liquid.

6. The device according to one claim 1, wherein it has a control (12) for the fluid pump (8).

7. The device according to claim 1, wherein it has a fluid (5) with CO₂-binding substances such as perfluorocarbons.

8. The device according to claim 1, wherein the CO₂ removal arrangement (7) is an oxygenator.

9. The device according to claim 1, wherein the CO₂ removal arrangement (7) has an oxygen enrichment arrangement.

10. The device according to claim 1, wherein the points of access (3, 4) to the abdominal wall are implantable, closable channels.

11. The device according to claim 1, wherein the points of access (3, 4) to the abdominal wall are permanently elastic, biocompatible sockets for superficial stitching or gluing.

12. The device according to claim 1, wherein the points of access (3, 4) to the abdominal wall are peritoneal dialysis catheters.

13. The device according to claim 1, wherein the points of access (3, 4) to the abdominal wall are tubes with lateral openings.

14. The device according to claim 1, wherein at least two points of access (3, 4) to the abdominal wall are constructed such that they are able to be arranged in one abdominal wall channel.

15. A method for operating a device according to claim 1, wherein a fluid (5) is directed through the fluid pump (8), the points of access (3, 4) to the abdominal wall and a CO₂ removal arrangement (7), in order to remove CO₂ from a fluid (5).

16. A method for the use of points of access (3, 4) to the abdominal wall, in which a fluid (5) is pumped through the points of access (3, 4) to the abdominal wall, wherein the fluid (5) is directed through a CO₂ removal arrangement (7).
